(19) United States
(12) Patent Application Publication
(10) Pub. No.: US 2011/0064847 A1
(43) Pub. Date: Mar. 17, 2011

(54) METHOD OF DENATURING PROTEIN WITH ENZYMES

(75) Inventors: Noriko Miwa, Kanagawa (JP); Nobuhisa Shimba, Kanagawa (JP); Mina Nakamura, Kanagawa (JP); Eiichiro Suzuki, Kanagawa (JP); Keiichi Yokoyama, Kanagawa (JP); Hiroyuki Nakagoshi, Kanagawa (JP); Fumiyuki Hirose, Kanagawa (JP); Hiroaki Sato, Kanagawa (JP)

(73) Assignees: AJINOMOTO CO., INC., Tokyo (JP); Amano Enzyme Inc., Nagoya-shi (JP)

(21) Appl. No.: 12/881,466

(22) Filed: Sep. 14, 2010

Related U.S. Application Data
(63) Continuation of application No. PCT/JP09/54792, filed on Mar. 12, 2009.
Application No. PCT/JP09/54792 is a continuation of application No. PCT/JP2009/294890, filed on Mar. 12, 2009.

(30) Foreign Application Priority Data
Mar. 14, 2008 (JP) 2008-066765
Nov. 18, 2008 (JP) 2008-294890

(51) Int. Cl. A23J 3/34; A23J 3/16; A23J 3/18; A23J 3/04; A23J 3/08; A23J 1/00; G01N 33/02; A23J 3/06

(52) U.S. Cl. 426/41; 426/7; 426/42; 426/46; 426/52; 426/56; 426/656; 426/63; 426/231

(57) ABSTRACT

A method of denaturing a protein by treating the protein with a protein glutaminase and a transglutaminase, a food containing a protein having been denatured with these enzymes, and an enzyme preparation for denaturing a protein which contains these enzymes. A protein is denatured by adding protein glutaminase and transglutaminase to the protein substantially at the same timing, or adding protein glutaminase to the protein before the transglutaminase acts on the protein, or controlling the quantitative ratio of protein glutaminase to transglutaminase, by which a protein is treated, to a definite level.

### CROSS-REFERENCE TO RELATED APPLICATIONS

This application is a continuation of PCT/JP2009/294890, filed on Mar. 12, 2009, and claims the benefit of the priority of Japanese patent application No. 2008-066765 filed on Mar. 14, 2008 and Japanese patent application No. 2008-294890 filed on Nov. 18, 2008, the disclosures of which are incorporated herein in their entirety by reference thereto.

### BACKGROUND OF THE INVENTION

1. Field of the Invention

The present invention provides a method of modifying a protein by treating the protein with both of transglutaminase and protein glutaminase, which are enzymes for modifying a glutamine residue in protein, and causing two enzyme reactions. The present invention also provides to a food containing a protein having been modified by the method, an enzyme preparation for modifying a protein which contains both of the protein glutaminase and transglutaminase in a specified ratio, and a method of establishing the optimum quantitative ratio of protein glutaminase to transglutaminase both of which are to be added to substrate protein.

2. Discussion of the Background

A transglutaminase (also referred to as “TG” hereinafter) is an enzyme that catalyzes an acyl transfer reaction between a γ-carboxyamide group of a glutamine residue and a primary amine in proteins and peptides. The TG acts on ε-amino group of lysine residue in protein as an acyl receptor, which results in cross-linking polymerization reaction of proteins. When a primary amine does not exist, water functions as an acyl receptor and the TG catalyzes deamidation reaction to transform a glutamine residue to a glutamic acid residue. The cross-linking reaction of proteins is mainly utilized for food treatment. Nowadays various TG preparations are developed for various foods such as kamaboko, ham, sausage, noodles, bean curd and bread by utilizing their characteristics of adding or improving of gel formability of protein, gelation of unheated protein or adhesiveness.

On the other hand, a protein glutaminase (also referred to as “PG” hereinafter) is an enzyme that has a function of deamidation of an amide group of a glutamine residue in protein. The function is disclosed in detail in, for example, Japanese Patent Kokai Publication No. JP-P2000-50887A, Japanese Patent Kokai Publication No. JP-P2001-218590A, and Yamaguchi et al., Eur. J. Biochem. Vol. 268, 2001, pp. 1410-1412. According to these documents, PG acts on the amide group in protein directly, which results in a transformation of glutamine residue into glutamic acid residue, and therefore an increasing of negative charge, an increasing of electrostatic repulsive force, a decreasing of isoelectric point, an increasing of hydration capability, etc. of protein occur because a carboxylic group is produced. As a result, improvements of functionalities such as an increasing of solubility and dispersion characteristic in water, an improvement of emulsification ability and emulsion stability, and the like are rendered and the broader usages of protein can be expected.

### SUMMARY OF THE INVENTION

As explained above, both of TG and PG are known as industrially useful enzymes; however, few or no concrete examples are reported to obtain new added values by combined use of these enzymes. Rather than that, Japanese Patent Kokai Publication No. JP-P2000-50887A discloses an example of use of PG as a TG reaction control agent and Newest Technology and Application of Food Enzyme Chemistry-Prospect of Proteomics-, CMC Publishing Co., Ltd., pp 146-153 discloses that a cross-linking reaction by TG does not proceed when PG and TG coexist.

A target of both enzymes in substrate protein is a glutamine residue, which is common to both of the enzymes. However, a specificity of PG to individual glutamine residue in protein is broader than that of TG. The reason is estimated that the TG requires ε-amino group of lysine residue as well as a glutamine residue, on the other hand, the PG requires only water which exists abundantly in a reaction environment other than a glutamine residue. For example, the PG has a far higher catalytic efficiency of reactivity to glutamine residue in casein than that of TG by judging from Km value and kcat value. When both TG and PG coexist, the reaction with PG occurs under priority to TG and the cross-linking reaction does not proceed because deamidated glutamine residue is no longer a target substrate for TG. Actually, by an experiment of adding PG during a cross-linking polymerization of casein by TG, it was proved that the polymerization of casein was ceased at the same time of addition of PG.

Moreover, it is confirmed that casein fully deamidated by PG is no longer a target substrate of TG, that is, no longer cross-linking-polymerized (Newest Technology and Application of Food Enzyme Chemistry-Prospect of Proteomics-, CMC Publishing Co., Ltd., pp 146-153). In addition, Y. S. Gu, et al. searched reactivity of the enzyme to an α-lactalbumin and reported that four of six glutamine residues were deamidated (Y. S. Gu et al., J. Agric. Food Chem. Vol. 49, 2001, pp. 5999-6005). On the other hand, they reported that substrate specificity of PG was broader than that of TG because actinomycete TG acted only on glutamine 54 which was one of the four glutamine residues. Japanese Patent Kokai Publication No. JP-P2000-50887A refers to a possibility that a known TG inhibitor such as an EDTA or ammonium chloride, which is not desirable for food additives, may be replaced by PG because the PG can cease the TG reaction at an appropriate point of time by taking advantage of high reactivity of PG to glutamine residue.

However, heretofore, no suggestion of combined use of TG and PG has been set forth with an objective other than the stopping of TG reaction by PG. Furthermore, the state of the art does not define conditions for obtaining desirable target effects by treating substrate protein with both of TG and PG, in more detail, a condition that the TG reaction is not ceased by PG and the TG reaction and its modifying effects is maintained even when PG coexists. Also, no detailed method is disclosed for effective use of both enzymes including what kinds of effects will be obtained when various kinds of substrate proteins are treated with TG and PG at various ratios. Therefore, at present, it is necessary to try and fail to find the best reaction conditions for each usage when developing protein products modified by TG and PG, which is regarded as completely a new attempt.

The enzyme reaction differs in its reaction amount and its degree of effect by various factors such as a treating time, temperature, amount of added enzyme, sort of substrate, condition, substrate specificity, and the like and it is difficult to use the two enzymes of common substrate efficiently. Therefore, much time and effort is need for a developer or manufacturer to determine manufacturing conditions of intended food for each use. That is because an enzyme reaction differs in its reaction amount and its degree of effect by various factors such as a treating time, temperature, amount of added enzyme, sort of substrate, condition, substrate specificity, and the like, it is necessary to research every combination when attempting to use two enzymes at the same time. Therefore, a method to use two enzymes of common substrate efficiently is desired.

At present, a method to use NMR is known for searching substrate specificity of TG (Japanese Patent Kokai Publication No. JP-P2002-332295A). This is a method to chase a TG reaction by treating any protein with TG under existence of ¹⁵N labeled ammonium chloride and detecting the ¹⁵N labeled nitrogen of carboxyamide of glutamine residue as a substrate of TG using NMR. The method can analyze both reactivity and substrate specificity at the same time using a protein substrate. However, Japanese Patent Kokai Publication No. JP-P2002-332295A does not disclose that the nitrogen of carboxyamide of glutamine residue that react with PG is labeled with ¹⁵N.

Therefore, it is an object of the present invention to provide a method of modifying a protein by treating the protein with both of protein glutaminase and transglutaminase, a food containing a protein modified with both of the enzymes, an enzyme preparation containing both of the enzymes for modifying a protein, and a method of easily establishing the optimum quantitative ratio of protein glutaminase to transglutamoinase.

Despite the expectation that the coexistence effect of PG and TG is difficult to obtain because TG does not work under the presence of PG, the inventors have found that both enzymes act on a substrate protein under certain conditions and a coexistence effect of PG and TG can be obtained even when both of the enzymes coexist. In addition, the inventors have found that PG has a function of catalyzing an exchanging reaction of glutamine residue in a protein with an ammonium salt as well as catalyzing a deamidation of the glutamine residue as the function of TG. That is, carboxylamide nitrogen of glutamine residue on which the TG and PG act is labeled with ¹⁵N. Therefore, the difference of reactivity can be determined from estimation of labeled rate with ¹⁵N by NMR signal intensity of ¹H-¹⁵N HSQC measurement, for example, that detects labeled ¹⁵N. The inventors have also found that an existence ratio of TG to PG has a good correlation with NMR signal intensity ratio for various proteins.

Moreover, the inventors have found that a quantitative ratio or activity ratio of PG to TG by which a signal intensity ratio of PG to TG (referred to as “PG/TG signal intensity ratio” hereinafter) within the range from 0.2 to 3.0 is the condition by which the TG reaction and its modifying effect can be maintained without ceasing of the TG reaction by PG in spite of coexistence of PG. Thus, the inventors have found that a condition for obtaining coexistence effect of both enzymes can be ascertained by treating a substrate with TG or PG under existence of ¹⁵N labeled ammonium and comparing substrate specificity of each enzyme using NMR. The inventors have found the merits of co-treatment with TG and PG at the same time and succeeded to establish a method of determining the condition easily and practically.

In view of the foregoing, the following illustrates certain embodiments of the present invention:

(1) A method of modifying a protein by treating the protein with both of protein glutaminase and transglutaminase in which a timing of adding protein glutaminase to a protein is essentially the same as a timing of adding transglutaminase to the protein or before the transglutaminse acts on the protein.

(2) The method of (1) described above in which a ratio by activity of the protein glutaminase to the transglutaminase, both of which are to be added to the protein, is described as the protein glutaminase: the transglutaminase=0.05 to 3:1.

(3) The method of (1) or (2) described above in which a ratio by weight of the protein glutaminase to the transglutaminase, both of which are to be added to the protein, is described as the protein glutaminase: the transglutaminase=0.01 to 0.7:1.

(4) The method of one of (1) to (3) described above in which a ratio by NMR signal intensity of the protein glutaminase to the transglutaminase, both of which are to be added to the protein, is described as the protein glutaminase: the transglutaminase=0.2 to 3.0:1.

(5) The method of one of (1) to (4) described above in which the protein is one or more selected from a group consisting of a milk protein, whey protein, soybean protein, wheat gluten, plasma, muscle protein, collagen and gelatin.

(6) A food comprising a protein modified by one of the method (1) to (5) described above.

(7) An enzyme preparation for modifying a protein comprising protein glutaminase and transglutaminase in which a ratio by activity of the protein glutaminase to the transglutaminase in the enzyme preparation is described as the protein glutaminase: the transglutaminase=0.05 to 3:1.

(8) An enzyme preparation for modifying a protein comprising protein glutaminase and transglutaminase in which a ratio by weight of the protein glutaminase to the transglutaminase in the enzyme preparation is described as the protein glutaminase: the transglutaminase=0.01 to 0.7:1.

(9) A enzyme preparation for modifying a protein comprising protein glutaminase and transglutaminase in which a ratio by NMR signal intensity of the protein glutaminase to the transglutaminase in the enzyme preparation is described as the protein glutaminase: the transglutaminase=0.2 to 3.0:1.

(10) A method of establishing the optimum ratio of protein glutaminase to transglutaminase, both of which are to be added to a protein, comprising a step of treating a protein with transglutaminase and protein glutaminase separately under presence of an isotope-labeled ammonium salt, a step of labeling a functional group of glutamine residue of the protein with the isotope, and a step of measuring an NMR signal intensity of the protein.

The present invention provides a new method of modifying a protein by which an effect of modifying a protein, which is a combined use effect of TG and PG and different from that when the TG or PG is used by alone, can be obtained. In detail, characteristics of a protein exerting to a food containing the protein are modified and an oral sensation (hardness, smoothness, and the like) of the food is improved. The present invention also provides a food containing a protein having new characteristics that can be obtained by the modifying method. Also the present invention provides a simple method of establishing the optimum condition (condition of adding) for obtaining the effect above explained by treating a protein with both of TG and PG that act on a substrate of the protein.

The above objects and embodiments highlight certain aspects of the invention. Additional objects, aspects and embodiments of the invention are found in the following detailed description of the invention.

### BRIEF DESCRIPTION OF THE FIGURES

A more complete appreciation of the invention and many of the attendant advantages thereof will be readily obtained as the same becomes better understood by reference to the following Figures in conjunction with the detailed description below.

FIG. 1 shows a ¹H-¹⁵N HSQC NMR spectrum of α-Lactalbumin in the presence of ¹⁵NH₄Cl (Experimental example 1).

### DETAILED DESCRIPTION OF THE INVENTION

Unless specifically defined, all technical and scientific terms used herein have the same meaning as commonly understood by a skilled artisan in enzymology, biochemistry, cellular biology, molecular biology, and food products.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will control. Further, the materials, methods, and examples are illustrative only and are not intended to be limiting, unless otherwise specified.

In the present invention, the transglutaminase may be one of a wide variety used for manufacturing foods such as gelatin, cheese, yoghurt, bean curd, steamed fish paste, ham, sausage, noodles, etc. and for improving quality of meat, and the like (Japanese Patent Kokai Publication No. JP-S64-27471A). In addition, the TG is an enzyme used for various industrial purposes such as a material for a microcapsule that is stable in heat, manufacturing a carrier of an immobilized enzyme, and the like. TG catalyzes an acyl transfer reaction of a γ-carboxyamide group of glutamine residue in a peptide chain of a protein molecule. When TG acts on ε-amino group of lysine residue of a protein molecule as an acyl receptor, an ε-(γ-glutamic acid)-lysine bonding is formed in and inter protein molecules

There are two types of TG, calcium independent type and calcium dependent type, and both types of TG may be used for the present invention. TG obtained from microorganisms such as an actinomycete or Bacillus subtilis, etc. may be an example as the former (for example, see Japanese Patent Kokai Publication No. JP-S64-27471A). Examples as the latter are exemplified as that obtained from a liver of a guinea pig (for example, see Japanese Patent Kokoku-examined Publication No. JP-H01-50382B), TG of human epidermal keratin cell (Phillips, M. A. et al. (1990) Proc. Natl. Acad. Sci. U.S.A., 87, 9333), human blood coagulation factor XIII (Ichinose, A. et al. (1990) Biochemistry 25, 6900), ones obtained from microorganisms such as an oomycete, etc., ones obtained from animal such as cattle blood or pig blood, etc., ones obtained from fish such as a salmon or porgy, etc. (for example, Nobuo Seki, et al., Nippon Suisan Gakkaishi (Bulletin of Japanese Society of Fisheries Science), vol. 56, pp 125-132, 1990), ones obtained from an oyster, and the like. Moreover, TG produced by genetic recombination (for example, Japanese Patent Kokai Publication No. JP-H01-300889A, Japanese Patent Kokai Publication No. JP-H06-225775A, and Japanese Patent Kokai Publication No. JP-H07-23737A), etc. can be applicable.

Any type of TG can be applicable to the present invention and not limited by its origin or production method. In a case where an enzyme reaction in a solvent containing calcium is not desirable due to characteristics of a protein labeled with an isotope, the calcium non-dependent TG is preferable for such a protein. For example, (MTG) obtained from microorganisms (Japanese Patent Kokai Publication No. JP-S64-27471A, for example) or the like satisfies the condition and therefore, it may be the best choice at the present time. They are, for example, obtained from Streptoverticillium griseocarneum, IFO 12776, Streptoverticillium cinnamoneum sub sp. Cinnamoneum, IFO 12852, Streptoverticillium mobaraense, IFO 13819, and the like. Transglutaminse obtained from Streptoverticillium mobaraense may be referred to as MTG hereinafter.

An “activity unit” of TG used in the present invention is determined and defined as follows. Benzyloxycarbonyl-L-Glutamylglycine (Z-Gln-Gly) and hydroxylamine as substrates are reacted with TG, and produced hydroxamic acid is converted into an iron complex in the presence of trichloroacetate and the amount of the iron complex is determined by absorbance at 525 nm. A calibration curve is obtained from the amount of the hydroxamic acid and an amount of enzyme that produces 1 μmol of hydroxamate per 1 min is defined as 1 unit of activity unit. The detailed method of the measurement is already disclosed (for example, Japanese Patent Kokai Publication No. JP-S64-27471A, etc.).

The PG used in the present invention acts directly to an amide group of a protein and causes deamidation without hydrolysis of peptide bonding and cross-linking of the protein. A kind of PG is not limited to the extent that the PG possesses such a function. Japanese Patent Kokai Publication No. JP-P2000-50887A and Japanese Patent Kokai Publication No. JP-P2001-218590A disclose such kinds of enzymes but not limited to those enzymes. PG prepared from a culture liquid for microorganism that produces the PG can be used. The microorganism for preparation of PG is not particularly limited and microorganisms such as Chryseobacterium, Flavobacterium and Empedobacter are illustrated.

Publicly known separation and purification methods of protein (such as centrifuging, UF concentration, salting-out, various kinds of chromatography with ion-exchanging resin, etc.) can be used for a preparation method of PG from a culture liquid for microorganism. For example, culture liquid is centrifuged to separate bacterial cells and then salting-out and chromatography, etc. may be combined to obtain target enzymes. When collecting enzymes from bacterial cells, the bacterial cells are crushed by pressure processing or supersonic processing, for example, and then separated and purified as described above to obtain target enzymes. Bacterial cells may be recovered from culture liquid by filtration or centrifuge, etc. prior to the processing steps above explained such as crushing of bacterial cells, separation and purification. The enzymes may be powdered by drying such as freeze drying or vacuum drying, etc. and appropriate diluent or drying auxiliary agent may be added at the drying step.

The “activity unit” of PG of the present invention can be measured by an improved method of a method of Japanese Patent Kokai Publication No. JP-P2000-50887A as follows.
(1) 10 μl of water solution containing PG is added to 100 μl of 176 mM phosphate buffer (pH 6.5) containing 30 mM of Z-Gln-Gly, incubated 10 minutes at 37 degrees C. and then the reaction is ceased by adding 100 μl of 12% TCA solution. At this time the solution is diluted with 20 mM phosphate buffer (pH 6.0) such that the concentration of enzyme becomes 0.05 mg/ml.
(2) The solution is centrifuged (12000 rpm, 4° C., 5 minutes) and NH₃ in the supernatant is measured with F-kit-Ammonia (Roche). The method is as follows.
(3) 10 μl of the supernatant and 190 μl of 0.1M triethanolamine buffer (pH 8.0) are added to 100 μl of liquid reagent II (attachment of the F-kit) and settled for 5 minutes at room temperature. After that an absorbance (E1) at 340 nm is measured using 100 μl of the solution. 1.0 μl of reagent III (glutamate dehydrogenase) is added to the remaining 200 μl of the solution and settled for 20 minutes at room temperature and then an absorbance (E2) at 340 nm is measured using the 200 μl of the solution. An ammonia concentration in the reaction solution is determined using a calibration curve indicating the relation between an ammonia concentration and variation of absorbance (at 340 nm) prepared using an ammonia standard solution attached to the F-kit.
(4) Concentration of protein is measured using protein assay CBB (Coomassie Brilliant Blue) solution (Nacalai Tesque) at 595 nm wavelength. BSA (Pierce) is used as a standard.
(5) Activity of PG is calculated by the following formula.

Relative activity (U/mg)=(ammonia concentration in reaction solution (μmol/ml)×volume of reaction solution (ml)×dilution rate of enzyme)/(enzyme amount (ml)×concentration of protein (mg/ml)×reaction time (min))

According to the present invention, a timing of adding PG to a protein is essentially the same as a timing of adding TG to the protein or before the TG acts on the protein. Therefore, the method disclosed in Japanese Patent Kokai Publication No. JP-P2000-50887A of ceasing TG reaction by adding PG is not included in the present invention because the timing of adding PG to a protein is after the TG acts on the protein. It should be noted that “PG and TG are added to a protein essentially at the same time” means that the PG and TG are added during a sequence of steps for addition of raw materials. In commercial manufacturing steps, raw materials are usually added sequentially, that is, at first material A is added, then material B is added, then material C is added, and so on instead of mixing these materials in advance. In such a case, when PG and TG are added during the sequential steps for addition of raw materials, the method is categorized that “protein glutaminase and transglutaminase are added to a protein essentially at the same time” as the present invention. It is needless to say, of course, in a case where raw materials including PG and TG are mixed in advance and then added, the method is categorized as “added essentially at the same time”.

Temperature needed for the reactions of both PG and TG generally ranges approximately from 3 to 60° C. and it will take about 1 minute to about 48 hours to progress the reactions. However, it may be better to spend about 5 minutes to about 24 hours at a temperature approximately, 5 to 50° C. When adding PG and TG to a food, only PG and TG may be added to a material containing a protein as a substrate, or PG and TG may be added with other materials. Amount of PG and TG to be added can be varied according to a kind of protein to be modified, final product or an effect to be obtained. For example, a standard amount of TG to be added is 0.1 to 100 units per 1 gram-weight of protein in materials for food. On the other hand, a standard amount of PG to be added is 0.01 to 120 units per 1 gram-weight of protein in materials for food. These amounts to be added are mere estimations and not limited to the values as far as the effect of the present invention can be obtained.

According to the present invention, a ratio of PG to TG, both of which are to be acted on a protein, is important. In a case where an adding timing of PG to a protein is essentially the same as an adding timing of TG to the protein or before the TG acts on the protein, the protein can be modified when the ratio of protein glutaminase to transglutaminase both of which are added to the protein is PG:TG=0.05 to 3:1 by activity and preferably PG:TG=0.05 to 2:1 because both of the PG and TG act on the protein in these range of the ratio. Or when the ratio of PG to TG is PG:TG=0.01 to 0.7:1 by weight, preferably PG:TG=0.01 to 0.4:1 and more preferably PG:TG=0.01 to 0.3:1, the protein can be modified because both of the PG and TG act on the protein in these range of the ratio. When the ratio of PG to TG is greater than the ratio above indicated, an action of the PG becomes too large compared to that of TG and the effect of using them together cannot be obtained. On the other hand, when the ratio is smaller, an action of PG is too small to obtain the effect of using them together.

When the ratio of protein glutaminase to transglutaminase both of which are added to the protein is PG:TG=0.2 to 3.0:1 by signal intensity of NMR and preferably PG:TG=0.2 to 2.3:1, the protein can be modified because both of the PG and TG act on the protein regardless to the adding timings. When the ratio of PG to TG is greater than the ratio above, an action of the PG becomes too large compared to that of TG and the effect of using them together cannot be obtained. On the other hand, when the ratio is smaller, an action of PG is too small to obtain the effect of using them together.

The ratio of signal intensity of NMR of the present invention can be calculated by the following method. A substrate protein is treated by PG or TG in the presence of isotope labeled (¹⁵N or ¹⁴N) ammonium salt to label a glutamine residue of the protein, and then an NMR signal of the labeled protein is measured and calculated. A kind of NMR measurement method is not limited and, for example, a correlation spectrum of ¹H and ¹⁵N such as a HSQC spectrum may be measured for detecting a glutamine residue labeled with ¹⁵N. When two or more signals are obtained, the sum of signal intensity is calculated as the signal intensity. As a result, the signal intensity ratio is expressed as “the sum of signal intensity when the protein is acted by PG”/“the sum of signal intensity when the protein is acted by TG”. The labeled compound may be an ammonium salt such as ammonium chloride and ammonium sulfate, and the like. When labeling the glutamine residue with ¹⁵N, an ammonium salt having ¹⁵N as ammonium nitrogen may be used and when labeling the glutamine residue with ¹⁴N, an ammonium salt having ¹⁴N as ammonium nitrogen may be used. A labeling method is such that a protein to be labeled and an ammonium salt are allowed to stand in an aqueous solvent at a pH ranging from about 3.0 to about 9.0 and preferably from about 4.0 to about 8.0 and at a temperature ranging from about 4 to about 65 degrees C. and preferably from about 25 to about 60 degrees C. A reaction time is not particularly limited and may be about 30 seconds to 1 week and preferably about 1 minute to about 1 day. In this reaction, a concentration of the ammonium salt may be preferably more than about ten times to a concentration of a protein to be labeled and more preferably more than about 200 times. When a concentration of the protein to be labeled is from about 1 μM to about 40 mM, a concentration of the ammonium salt may be preferably from about 10 μM to about 10 M. The signal intensity ratio of the present invention can be measured for every kind of food containing protein because proteins contained in food such as α-lactoglobulin, casein, soybean globulin, myosin, actomyosin, and the like can be labeled with isotope.

An example is explained that a protein is reacted with isotope-labeled ammonium chloride by treating with TG or PG. When α-lactalbumin (referred to as “α-La” hereinafter) is used as a substrate, a solution of the substrate (10 mg/ml of α-La, 200 ml of ¹⁵NH₄Cl, 5% D₂O/20 mM Tris-HCl (pH 7.0)) is prepared. Then TG or PG is added such that a ratio of substrate to enzyme becomes 1000:1 and incubated for 26.5 hours at 37° C. The incubated solution is poured into an NMR sample tube and ¹H-¹⁵N HSQC is measured using Avance 600 (Bruker Corporation), etc. Carboxyamide nitrogen is replaced with ¹⁵N and two signals are observed as a pair per one chemical shift of ¹⁵N because two atoms of ¹H are bonded with the ¹⁵N. The ratio of NMR signal intensity can be calculated by “the sum of signal intensity treated with PG”/“the sum of signal intensity treated with TG”.

A protein that can be modified by the present invention is not particularly limited and, for example, milk protein, whey protein, soybean protein, wheat gluten, muscle protein, plasma, collagen, gelatin, and the like may be used. A mixture of two or more of these proteins may be also possible. A food according to the present invention is not limited by kinds of raw material or processed state as far as it contains the protein above mentioned modified by an use of PG and TG together. A food of processed state such as, for example, sterilized state, defatted state, diluted state, condensed state, dried state, and the like is included within the scope of the present invention.

Next, an enzyme preparation of the present invention will be explained. An enzyme preparation of the present invention for modifying a protein has a condition that a mixing ratio of transglutaminase to protein glutaminase as essential components is in the range from PG:TG=0.05 to 3:1 by activity and preferably from PG:TG=0.05 to 2:1; in the range from PG:TG=0.01 to 0.7:1 by weight, preferably from PG:TG=0.01 to 0.4:1 and more preferably from PG:TG=0.01 to 0.3:1; or in the range of PG:TG=0.2 to 3.0:1 by NMR signal intensity and preferably from PG:TG=0.2 to 2.3:1. Any component that is generally used in the present field other than PG and TG such as lactose, sucrose, maltitol, sorbitol, dextrin, branched dextrin, cyclodextrin, starch, polysaccharides, gum, pectin, and the like may be formulated. An animal protein or a vegetable protein such as a soybean protein, wheat protein, etc. can be also formulated. Moreover, an inorganic salt physiologically acceptable such as a sodium bicarbonate, sodium citrate, sodium phosphate, sodium chloride, potassium chloride, etc. can be formulated when necessary in the enzyme preparation of the present invention. Moreover, a seasoning, sugar, spice, coloring agent, color coupler, organic salt such as an ascorbic acid and its salt, or an emulsifier, oil and fat can be formulated as necessary.

The present invention includes a simple method to find an optimum quantitative adding ratio of protein glutaminase to transglutaminase. The method is that, as already described, a protein is treated with PG and TG separately in the presence of an isotope labeled ammonium salt, to label a functional group of a glutamine residue of the protein with the isotope, and the NMR signal intensity is measured. The optimum quantitative adding ratio of PG to TG can be established by finding a condition that the NMR signal intensity ratio of protein glutaminase to transglutaminase becomes from protein glutaminase:transglutaminase=0.2 to 3.0:1 and preferably from 0.2 to 2.3:1. The optimum range of the NMR signal intensity ratio of PG to TG ranges from protein glutaminase:transglutaminase=0.2 to 3.0:1 and preferably ranges from 0.2 to 2.3:1 without depending on kinds of substrate proteins. Because the NMR signal intensity ratio correlates to the optimum adding ratio (activity ratio or weight ratio) of PG to TG for each of the substrate protein, the optimum adding ratio (activity ratio or weight ratio) of PG to TG can be easily found without repeating trial and error of many experiments.

The above written description of the invention provides a manner and process of making and using it such that any person skilled in this art is enabled to make and use the same, this enablement being provided in particular for the subject matter of the appended claims, which make up a part of the original description.

As used herein, the phrases “selected from the group consisting of,” “chosen from,” and the like include mixtures of the specified materials.

Where a numerical limit or range is stated herein, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

Having generally described this invention, a further understanding can be obtained by reference to certain specific examples, which are provided herein for purposes of illustration only, and are not intended to be limiting unless otherwise specified.

### EXAMPLES

### Experimental Example 1

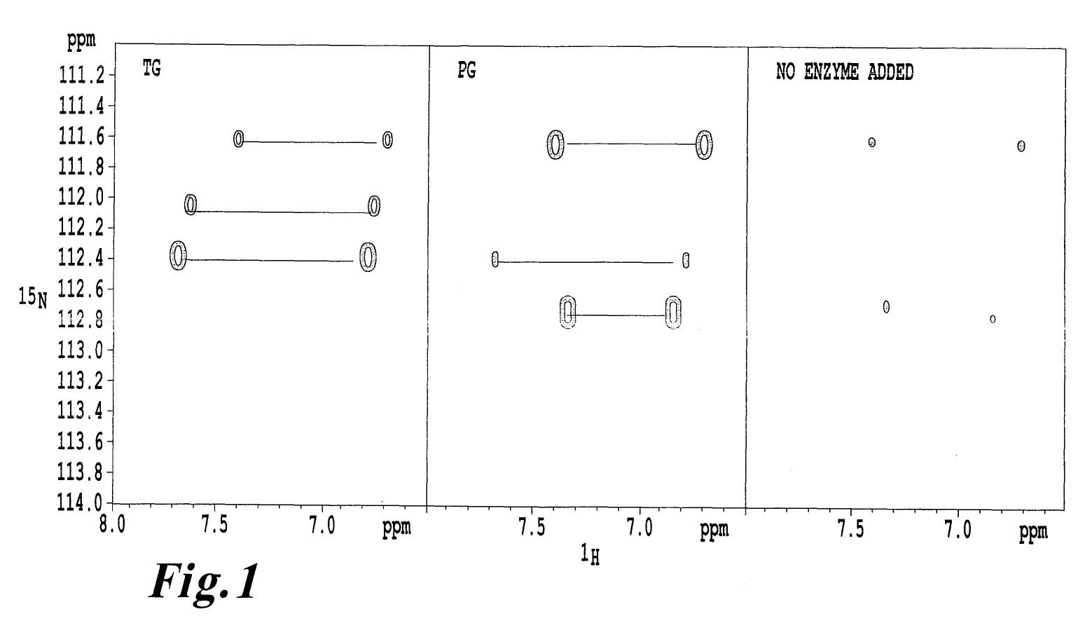

An α-lactalbumin (referred to as “α-La” hereinafter) (Sigma) was used as a substrate. A solution of the substrate (10 mg/ml of α-La, 200 ml of ¹⁵NH₄Cl, 5% D₂O/20 mM Tris-HCl (pH 7.0)) was prepared and then TG (purified enzyme from “Activa” TG, Ajinomoto Co., Inc.) or PG (prepared from Chryseobacterium described in Patent Document 1) was added such that a ratio of substrate to enzyme of 1000:1 was achieved. The mixture was incubated for 26.5 hours at 37° C. The incubated solution was poured into an NMR sample tube and ¹H-¹⁵N HSQC was measured using NMR (Avance 600, Bruker Corporation). The result of the ¹H-¹⁵N HSQC measurement after 26.5 hours is shown in FIG. 1.

When carboxyamide nitrogen is replaced with ¹⁵N, two signals are observed as a pair per one chemical shift of ¹⁵N because two atoms of ¹H are bonded with the ¹⁵N. It can be seen that a part of nitrogen atoms of carboxyamides of glutamine residues in α-La is labeled with ¹⁵N and succeeded in exhibiting ¹⁵N labeling by PG as shown in FIG. 1. Therefore, it was proved that PG can also cause a reaction between ammonium chloride and glutamine residue in addition to deamidation reaction likewise as TG. NMR signal intensity is indicated as the sum of areas of oval spots in FIG. 1 and the larger the area is, the larger the amount of the reaction is.

### Example 1

TG or PG as explained in Experimental Example 1 was added independently to a milk on the market, 0.2 M ¹⁵NH₄Cl and 5% D₂O, and signal intensity at each added concentration was measured. TG was added such that a ratio of substrate to enzyme (S/E ratio) was 7400/1 by weight. On the other hand, PG was added by 0.002 to 5 times of TG by weight. A relative activity of the TG was 26 units per 1 mg of enzyme protein and a relative activity of the PG was 120 units per 1 mg of enzyme protein. The solution containing the enzyme was then incubated for 3 hours at 37° C., and then poured into an NMR sample tube and measured by ¹H-¹⁵N HSQC using NMR described in Experimental Example 1. The amount of added PG was indicated by weight ratio (PG/TG) of enzymes and activity ratio (PG/TG) against the amount of TG added. Signal intensity ratio (PG/TG) corresponding to each ratio is shown in Table 1.

TABLE 1

| | | | | | Signal |
|---|---|---|---|---|---|
| | | Weight ratio | | | intensity |
| | | of enzymes | Activity ratio | Signal | ratio |
| TG | PG | (PG/TG) | (PG/TG) | intensity | (PG/TG) |
| 2.5 μg | — | — | — | 92.7 | — |
| — | 12.5 μg | 5 | 23 | 474 | 5.11 |
| — | 2.5 μg | 1 | 4.6 | 308.1 | 3.32 |
| — | 0.5 μg | 0.2 | 0.92 | 147 | 1.58 |
| — | 0.25 μg | 0.1 | 0.46 | 93.5 | 1 |
| — | 0.025 μg | 0.01 | 0.046 | 20.1 | 0.216 |

Yoghurt was made from milk on the market. 400 g of milk on the market was heated to 50° C. and added with TG and PG described in Experimental Example 1 by the amounts shown in Table 2.

TABLE 2

| | | | | Enzyme | Enzyme | |
|---|---|---|---|---|---|---|
| | | | | weight | activity | Signal |
| | | | | ratio | ratio | intensity |
| | | TG | PG | (PG/ | (PG/ | ratio |
| | enzyme | (mg) | (mg) | TG) | TG) | (PG/TG) |
| Control sample | — | — | — | — | — | — |
| Product 1 of | TG, PG | 1.15 | 0.0115 | 0.01 | 0.046 | 0.216 |
| the present | | | | | | |
| invention | | | | | | |
| Product 2 of | TG, PG | 1.15 | 0.023 | 0.02 | 0.092 | Not |
| the present | | | | | | measured |
| invention | | | | | | |
| Product 3 of | TG, PG | 1.15 | 0.115 | 0.1 | 0.46 | 1 |
| the present | | | | | | |
| invention | | | | | | |
| Product 4 of | TG, PG | 1.15 | 0.23 | 0.2 | 0.92 | 1.58 |
| the present | | | | | | |
| invention | | | | | | |
| Comparative | TG | 1.15 | — | — | — | — |
| product T | | | | | | |
| Comparative | PG | — | 1.15 | — | — | — |
| product P | | | | | | |
| Comparative | TG, PG | 1.15 | 1.15 | 1 | 4.6 | 3.32 |
| product T-P | | | | | | |

A relative activity of the TG was 26 units per 1 mg of enzyme protein and a relative activity of the PG was 120 units per 1 mg of enzyme protein. After enzyme reaction of 90 minutes at 50° C., it was heated in a boiling bath with stirring. As soon as the temperature reached 90° C., it was soaked in water with ice to cool to 45° C. 20 g (5% weight of original material) of starter (Bulgaria yoghurt, Meiji Dairies Corporation) was added, stirred completely and divided to each sample cup by 40 g. The cups were covered with aluminum foil and fermented approximately 3 to 4 hours at 38° C. until pH dropped to 4.5. They were reserved in low temperature (4° C.) and evaluated next day by a physical property test and a sensory test.

A breaking stress as a physical property was measured using a texture analyzer (Eko Instruments). A cylindrical plunger of 10 mm in diameter was used and a breaking speed was set at 6 cm/min (1 mm/sec). The result is shown in Table 3.

TABLE 3

| | Breaking stress |
|---|---|
| | (g) |
| Control sample | 18.63 |
| Product 1 of the present | 33.88 |
| invention | |
| Product 2 of the present | 31.74 |
| invention | |
| Product 3 of the present | 24.68 |
| invention | |
| Product 4 of the present | 20.51 |
| invention | |
| Comparative product T | 32.17 |
| Comparative product P | 12.91 |
| Comparative product T-P | 12.44 |

A breaking stress of a comparative product T, in which only TG was added, increased remarkably compared with a control sample. The result is attributed to an effect of linkage of milk protein by the TG. Products 1 to 4 of the present invention also showed relatively high breaking stresses compared with the control sample, which means that an effect to increase breaking stress by TG was maintained. However, because a breaking stress of a comparative product T-P does not differ from that of comparative product P, it was confirmed that the TG reaction was almost ceased by PG and therefore the effect by TG was not observed.

Hardness was evaluated as a sensory test by 6 panels. The hardness of the control sample is set as 0 point, and each product was scored by +5 points as hardest and −5 points as softest and an average point for each product was calculated. The result is shown in Table 4.

TABLE 4

| | Average | | Overall |
|---|---|---|---|
| | hardness | | evaluation |
| | (n = 6) | Comment | (point) |
| Control sample | 0 | — | 2.5 |
| Product 1 of the | 3.7 | hard, smooth texture | 3.1 |
| present | | | |
| invention | | | |
| Product 2 of the | 3.8 | hard, smooth texture | 3.2 |
| present | | | |
| invention | | | |
| Product 3 of the | 3.2 | very thick oral sensation, | 4.8 |
| present | | creamy | |
| invention | | | |
| Product 4 of the | 2.6 | thick oral sensation, creamy | 4.2 |
| present | | | |
| invention | | | |
| Comparative | 3.2 | hard and rough | 1.9 |
| product T | | | |
| Comparative | −2 | too soft | 2 |
| product P | | | |
| Comparative | −1.2 | too soft | 2.4 |
| product T-P | | | |

1 point: not favorable
2 points: not so favorable
3 points: fair
4 points: favorable
5 points: very favorable

The comparative product T, in which only TG was added, increased its hardness remarkably compared with the control sample. Products 1 to 4 of the present invention also kept increased hardness obtained by TG. However, the hardness of yoghurt of comparative product T-P was softened as the comparative product P, which means that the TG reaction was almost ceased by PG and therefore the effect by TG was not observed. The result roughly corresponds to the result of the physical property test explained above. The overall evaluation of oral sensation preference indicated that the products 1 to 4 of the present invention obtained favorable result, that is, the products kept an effect of increased hardness of yoghurt by TG and, on the other hand, smoothness was improved than the case when TG only was added. This means that an effect of using PG and TG together was confirmed.

### Example 2

Skim milk powder (low heat-type, milk protein 35%, Yotsuba Co., Ltd.) was added by 0.85% to Takanashi Milk Products Co. Ltd.'s low fat milk (milk protein 3.3%, milk fat 1.0%) to adjust the milk protein concentration to 3.6% and dissolved at 55 degrees C. by heating to prepare raw material milk for yoghurt. The raw material milk was heated in a boiling bath, kept in two minutes after reached 95° C. and then cooled in an ice bath immediately. When the raw material milk reached 47° C., a lactic acid bacteria starter (Yo-Flex, DVS YC-370, Christian Hansen) was added by 0.006%, and TG and PG were added according to Table 5 and stirred well. Then it was divided into plastic cups by specified amount and fermented in an incubator at 44° C. until the pH reached 4.5 to 4.6. It took approximately 4 to 5 hours from the beginning to the end of fermentation. After fermentation, the products were preserved in a refrigerator at 5° C. Next day, an amount of syneresis on the surface of the obtained set-type yoghurt was observed.

TABLE 5

| | | | Activity ratio | Weight ratio |
|---|---|---|---|---|
| | TG(u/gp) | PG(u/gp) | (PG/TG) | (PG/TG) |
| Control sample | 0 | 0 | — | — |
| Comparative | 0.5 | 0 | — | — |
| product 1 | | | | |
| Comparative | 0 | 0.5 | — | — |
| product 2 | | | | |
| Comparative | 0 | 1 | — | — |
| product 3 | | | | |
| Product 1 of the | 0.5 | 0.5 | 1 | 0.22 |
| present | | | | |
| invention | | | | |
| Product 2 of the | 0.5 | 1 | 2 | 0.44 |
| present | | | | |
| invention | | | | |

Physical properties of the yoghurt were evaluated using a texture analyzer (Stable Macro System, Ltd.). A sensory test was performed by 5 trained panels. A breaking stress and an adhesion area of the yoghurt were measured at a condition for the physical property measurement of 1 mm/sec velocity, plate plunger of 10 mm diameter and 10% of compression. The inventors have already confirmed that the breaking stress highly correlates to hardness of yoghurt and the adhesion area highly correlates to texture of creaminess of yoghurt. The result is shown in Table 6.

TABLE 6

| | syneresis | breaking | adhesion | | overall |
|---|---|---|---|---|---|
| | on surface | stress (g) | area (g * ε) | sensory test (n = 5) | evaluation |
| Control | large | 19.1 | 3.26 | rough surface, sloppy and | X |
| sample | | | | soft texture | |
| Comparative | none | 24.3 | 3.93 | glossy surface, hard and | X |
| product 1 | | | | crumbly (like kanten), but | |
| | | | | sloppy | |
| Comparative | large | 15.8 | 4.31 | rough surface, soft and | Δ |
| product 2 | | | | smooth | |
| Comparative | fairly | 14.3 | 4.52 | fairly rough surface, very | Δ |
| product 3 | large | | | soft and smooth, of less | |
| | | | | body | |
| Product 1 of | none | 28.6 | 4.41 | glossy surface, feel body | ◯ |
| the present | | | | and smooth | |
| invention | | | | | |
| Product 2 of | none | 21.1 | 5.2 | glossy surface, feel body, | ◯ |
| the present | | | | very smooth and creamy | |
| invention | | | | | |

A large amount of syneresis was observed on the surfaces of the control sample and comparative product 2 and a fairly large amount of syneresis was observed on the comparative product 3. No syneresis was observed on the surfaces of the comparative product 1 and products 1 and 2 of the present invention and the surfaces of these products were glossy. The physical property measurements and the sensory evaluation showed that the comparative product 1 had a hard and crumbly texture like an agar gel and was sloppy in a mouth. The comparative products 2 and 3 decreased their hardness than the control sample and increased their adhesiveness. The sensory test also caused a comment that they were smooth but soft and of less body. On the other hand, the products 1 and 2 of the present invention increased both of hardness and adhesiveness and they were of body, smooth and creamy yoghurt. As an overall evaluation including appearance, physical property and oral sensation, the products 1 and 2 of the present invention are the most favorable yoghurts, indicating that an improvement of oral sensation of set-type low fat yoghurt is possible by adding TG and PG in an appropriate balanced ratio of blending.

### Example 3

Skim milk powder (low heat-type, milk protein 35%, Yotsuba Co., Ltd.) was added to Takanashi Milk Products Co. Ltd.'s low fat milk (milk protein 3.3%, milk fat 1.0%), to adjust the milk protein concentration to 3.94% and dissolved at 55° C. by heating to prepare raw material milk for yoghurt. The raw material milk was heated in a boiling bath, kept in two minutes after reached 95° C. and then cooled in an ice bath immediately. When the raw material milk reached 47° C., a lactic acid bacterium starter (Yo-Flex, DVS YC-370, Christian Hansen) was added by 0.006% and TG and PG were added according to Table 7 and stirred well. Then it was divided into stainless cups by specified amount and fermented in an incubator at 44° C. until the pH reached 4.5 to 4.6. After fermentation, the products were cooled in a refrigerator at 5° C. and filtered through a filter of 216 μm meshes to make stirred yoghurt. The resultant stirred yoghurt was preserved at 5° C. and a change of appearance and oral sensation during preservation were evaluated.

TABLE 7

| | | | Activity | |
|---|---|---|---|---|
| | | | ratio | Weight ratio |
| | TG(u/gp) | PG(u/gp) | (PG/TG) | (PG/TG) |
| Control sample | 0 | 0 | — | — |
| Comparative | 0.6 | 0 | — | — |
| product 1 | | | | |
| Comparative | 0 | 0.6 | — | — |
| product 2 | | | | |
| Product 1 of the | 0.6 | 0.1 | 0.17 | 0.037 |
| present invention | | | | |
| Product 2 of the | 0.6 | 0.3 | 0.5 | 0.11 |
| present invention | | | | |
| Product 3 of the | 0.6 | 0.6 | 1 | 0.22 |
| present invention | | | | |
| Product 4 of the | 0.6 | 1.0 | 1.7 | 0.37 |
| present invention | | | | |

A physical property of the yoghurt was measured by a dynamic viscoelasticity measuring device (Rheostress RS1, HAAKE). A sensory test was performed by 3 trained panels. A measurement condition for physical property of the yoghurt was programmed, in which a shearing speed of a corn plate of 6 cm diameter was increased from zero to 100 (l/s) during 300 seconds and then the shearing speed was decreased from 100 to zero (l/s) during the same period of time. The measurement temperature was 10° C. and a viscosity at the shearing velocity 100 (l/s) was recorded. The result is shown in Table 8.

TABLE 8

| | | sensory test | | | oral | |
|---|---|---|---|---|---|---|
| | syneresis | immediately after | | lump after | sensation | |
| | on | trial preparation | Viscosity | long | after long | overall |
| | surface | (n = 5) | (mPεs) | preservation | preservation | evaluation |
| Control | Large | rough, sloppy and | 98.7 | None | not changed | X |
| sample | | soft texture | | | | |
| Comparative | None | rough and of body | 160.9 | Large | texture like | X |
| product 1 | | | | | sand | |
| Comparative | Small | glossy surface, | 99.3 | None | Not changed | Δ |
| product 2 | | smooth but sloppy | | | | |
| | | and soft | | | | |
| Product 1 | None | glossy surface, of | 161.5 | Fairly small | decreased | ◯ |
| of the | | body, fairly smooth | | | texture like | |
| present | | | | | sand | |
| invention | | | | | | |
| Product 2 | None | glossy surface, of | 157.8 | Small | no texture | ⊚ |
| of the | | body, very smooth | | | like sand | |
| present | | | | | | |
| invention | | | | | | |
| Product 3 | None | glossy surface, | 129.0 | None | no texture | ◯ |
| of the | | fairly of body, very | | | like sand | |
| present | | smooth | | | | |
| invention | | | | | | |
| Product 4 | None | glossy surface, of | 116.5 | none | no texture | ◯ |
| of the | | body than reference, | | | like sand | |
| present | | very smooth | | | | |
| invention | | | | | | |

Before preparation of the stirred yoghurts, a large amount of syneresis and small amount of syneresis was observed on the control sample and the comparative product 2, respectively, and almost no syneresis was observed on the comparative product 1 and products 1 to 4 of the present invention. According to the sensory test immediately after preparation of the stirred yoghurt, the control sample had rough, sloppy and soft texture. The comparative product 1 had body but felt rough, and the comparative product 2 had a glossy surface and felt smooth but sloppy and soft. The products 1 to 4 of the present invention had glossy surfaces and had smoothness as well as body.

The results of the viscosity measurement well correspond to the results of the sensory tests. That is, the viscosities of the products 1 to 4 of the present invention were apparently high compared with that of the control sample or the comparative product 2, which proved that the products 1 to 4 of the present invention have obtained body. The change of appearances and oral sensation after three weeks' preservation in a refrigerator was that lumps were produced in the comparative product 1 and rough texture like sand was strongly sensed. However, the texture was improved in the product 1 of the present invention and almost eliminated in the products 2 to 4 of the present invention. According to the results explained above in all, the products 1 to 4 of the present invention were evaluated to be apparently superior to the control sample and comparative products 1 and 2 by the points of the physical property and oral sensation (after preservation).

As explained above, the yoghurt added only by TG (comparative product 1) produces body compared with yoghurt added no TG but the quality degradation during preservation may be a problem. On the other hand, the yoghurt added only by PG (comparative product 2) feels smooth but the sloppy texture without body may be a problem. However, it was proved that the yoghurt added by TG and PG at a specified ratio (products 1 to 4 of the present invention) could improve quality of the yoghurt by rendering glossy surface and smoothness as well as eliminating above problems.

### Example 4

TG or PG described in EXPERIMENTAL EXAMPLE 1 were solely added to soya milk on the market (not modified, final protein content 4.8%, Taishi-Food Inc.), 0.2M ¹⁵NH₄Cl and 5% D₂O and mixed well. The TG was added such that a ratio of substrate/enzyme (S/E ratio) was 6000/1 by weight. The PG was added by 0.01 to 1 time of the amount of the TG. The solution was incubated for 1 hour and 15 minutes at 37° C. and then the solution was poured into an NMR sample tube and ¹H-¹⁵N HSQC was measured by the NMR described in EXPERIMENTAL EXAMPLE 1. Calculated results of signal intensity ratio of PG/TG at each enzyme weight ratio (PG/TG) and activity ratio (PG/TG) as is explained in EXPERIMENTAL EXAMPLE 1 are shown in Table 9.

TABLE 9

| | | | | | Signal |
|---|---|---|---|---|---|
| | | Enzyme weight | | | intensity |
| | | ratio | Activity ratio | Signal | ratio |
| TG | PG | (PG/TG) | (PG/TG) | intensity | (PG/TG) |
| 4 μg | — | — | — | 160.96 | |
| — | 4 μg | 1 | 4.6 | 373.48 | 2.52 |
| — | 1 μg | 0.25 | 1.15 | 364.31 | 2.26 |
| — | 0.4 μg | 0.1 | 0.46 | 208.87 | 1.29 |
| — | 0.2 μg | 0.05 | 0.23 | 113.22 | 0.703 |
| — | 0.04 μg | 0.01 | 0.046 | 23.421 | 0.145 |

Bean curds added by TG and PG by the ratio described in Table 10 were prepared. A solidification container containing 10 g of 30% bittern solution (containing 3 g of bittern, Ako Kasei Co., Ltd.) and a large stainless cup containing 1 kg of soya milk on the market above were heated to 55° C. in a hot water bath. An enzyme solution was added into the soya milk and stirred. The soya milk was poured immediately into the solidification container containing the bittern vigorously and a stirring plate was moved up and down by four times. The container was covered with a cap and then transferred into an incubator and allowed to stand for 50 minutes at 55° C. After that the bean curd was transferred into water in a vat and kept 30 minutes to 1 hour. The bean curd was divided and put into curd containers and weighed the content. Water was poured into the containers up to a level flush with the bean curd and heat-sealed with films. They were put into a thermostat water bath at 85° C. and kept for 45 minutes (secondary heating) and then cooled at first in a flowing water and then in an iced bath, and reserved in cold storage. Next day water was drained and the content in the container was weighed and physical property test and sensory test were performed.

TABLE 10

| | | | | Enzyme | Enzyme | |
|---|---|---|---|---|---|---|
| | | | | weight | activity | Signal |
| | | | | ratio | ratio | intensity |
| | | TG | PG | (PG/ | (PG/ | ratio |
| | Enzyme | (mg) | (mg) | TG) | TG) | (PG/TG) |
| Control sample | — | — | — | — | — | — |
| Comparative | TG | 0.15 | — | — | — | — |
| product (T1) | | | | | | |
| Comparative | TG | 0.75 | — | — | — | — |
| product (T2) | | | | | | |
| Comparative | PG | — | 0.036 | — | — | — |
| product (P1) | | | | | | |
| Comparative | PG | — | 0.18 | — | — | — |
| product (P2) | | | | | | |
| Comparative | TG, PG | 0.15 | 0.18 | 1.2 | 5.5 | Not |
| product (T-P) | | | | | | measured |
| Product 1 of the | TG, PG | 0.15 | 0.038 | 0.25 | 1.15 | 2.26 |
| present invention | | | | | | |
| Product 2 of the | TG, PG | 0.75 | 0.038 | 0.05 | 0.23 | 0.703 |
| present invention | | | | | | |
| Product 3 of the | TG, PG | 0.75 | 0.19 | 0.25 | 1.15 | 2.26 |
| present invention | | | | | | |

A breaking test was performed as a physical property test using a rheometer (Fudou Kougyou Inc.). The plunger was disc-shaped whose size was 5 mm in diameter and a breaking speed was 6 cm/min (1 mm/sec). A sensory test was performed by 5 panels and smoothness and hardness were evaluated such that a product without enzyme scored zero point, and each product was evaluated by 7 grades between ±3 points and an average was calculated. The result is shown in Table 11. The comparative products T1 and T2 in which TG was added increased their breaking stresses compared with the control sample.

On the other hand the comparative products P1 and P2 in which PG was added decreased their breaking stresses compared with the control sample. A breaking stress of the product 1 of the present invention was almost equivalent degree to that of the comparative product T1 in which the same amount of TG was added and the texture of the product 1 of the present invention changed smoother. A breaking stress of the product 2 of the present invention slightly decreased compared with the comparative product T2 in which the same amount of TG was added; however, the breaking stress was still large compared with the control sample and smoothness was also given. A breaking stress of the product 3 of the present invention decreased compared with the comparative product T2 in which the same amount of TG was added; however, the breaking stress was larger than that of the comparative product P2 in which the same amount of PG was added and therefore, an effect of combined use of TG was confirmed. The comparative product T-P was as smooth as that of comparative product P1 or P2 and a hardening effect of the TG reaction was not observed. The result means that the TG reaction was almost ceased by PG. The sensory test showed that the hardening effect by TG was observed in the products 1 to 3 of the present invention and their textures were improved compared with the case only TG was added. As an overall result, a preferable texture was obtained for the products of the present invention, which means that the effect of using PG and TG together was confirmed.

TABLE 11

| | Breaking | | Overall |
|---|---|---|---|
| | stress | | evaluation |
| | (g) | Comments | (point) |
| Control sample | 33.9 | — | 2.5 |
| Comparative | 38.4 | hard | 3.2 |
| product (T1) | | | |
| Comparative | 54.1 | very hard | 3.5 |
| product (T2) | | | |
| Comparative | 28.2 | soft | 2.4 |
| product (P1) | | | |
| Comparative | 17.9 | very soft, fragile (crumbly) | 2 |
| product (P2) | | | |
| Comparative | 21.5 | too soft | 2.7 |
| product (T-P) | | | |
| Product 1 of the | 36.6 | very smooth, creamy | 4.1 |
| present invention | | | |
| Product 2 of the | 49.2 | hard and smooth texture | 4.8 |
| present invention | | | |
| Product 3 of the | 34.1 | very smooth, creamy | 4.2 |
| present invention | | | |

1 point: not favorable
2 points: not so favorable
3 points: fair
4 points: favorable
5 points: very favorable

### Example 5

Japanese udon noodles and Chinese noodles were prepared according to the formulation and trial procedures shown in Table 12. Transglutaminase and protein glutaminase were dissolved in water when added. The amounts of enzymes added in the udon noodles and Chinese noodles are shown in Table 13. The udon noodles were boiled for 20 minutes in hot water 15 times the amount of 100 g of the noodle and the oral sensation was evaluated by trained panels (n=5). The Chinese noodles were evaluated as the udon noodles except for the boiling time of 5 minutes. Hardness, stickiness and smoothness were evaluated by the standard of a control sample (apparently decreased: x x, slightly decreased: x, equal: Δ, slightly increased: ◯, apparently increased: ⊚). Because the results of the evaluation for udon noodles and Chinese noodles had almost the same tendency, they are shown together in Table 14. When the ratio of PG/TG by weight was 0.01, 0.05 or 0.2 (corresponding to product 1, 2 or 3 of the present invention, respectively), the oral sensation was well balanced with hardness, stickiness and smoothness for both of the noodles. It was proved that these ratios were also appropriate ratios for noodles to obtain favorable effects of both transglutaminase and protein glutaminase by using them together.

TABLE 12

| | | |
|---|---|---|
| Udon | formulation | Wheat 100 portions, salt 3 portions, |
| noodles | | water 40 portions |
| | procedure | First rolling, scale 2.5→combined rolling, |
| | | scale 2.5→rolling, scale 2.3→rolling, |
| | | scale 2.1 →rolling, scale 1.7→rolling, |
| | | scale 1.5→ferment, 1 h →cutting (angular |
| | | blade #12) |
| Chinese | formulation | Wheat 100 portions, salt 1 portion, lye water A 1 |
| noodles | | portion, food coloring 0.1 portion, |
| | | water 36 portions |
| | procedure | First rolling, scale 1.5→combined rolling, |
| | | scale 1.5→rolling, scale 1.3→rolling, |
| | | scale 1.1 →rolling, scale 0.7→rolling, |
| | | scale 0.5→ferment, 1 h →cutting (angular |
| | | blade #22) |

TABLE 13

| | | | | | Enzyme |
|---|---|---|---|---|---|
| | | | | Enzyme | activity |
| | | TG | PG | weight ratio | ratio |
| Category | Enzyme | (mg) | (mg) | (PG/TG) | (PG/TG) |
| Control sample | — | 0 | 0 | — | — |
| Comparative | PG | 0 | 0.1 | — | — |
| product 1 | | | | | |
| Comparative | PG | 0 | 0.5 | — | — |
| product 2 | | | | | |
| Comparative | PG | 0 | 2 | — | — |
| product 3 | | | | | |
| Comparative | TG | 10 | 0 | — | — |
| product 4 | | | | | |
| Product 1 of | TG, PG | 10 | 0.1 | 0.01 | 0.046 |
| the present | | | | | |
| invention | | | | | |
| Product 2 of | TG, PG | 10 | 0.5 | 0.05 | 0.23 |
| the present | | | | | |
| invention | | | | | |
| Product 3 of | TG, PG | 10 | 2 | 0.2 | 0.92 |
| the present | | | | | |
| invention | | | | | |

TABLE 14

| | | | | Overall |
|---|---|---|---|---|
| Evaluation category | Hardness | Stickiness | Smoothness | evaluation |
| Control sample | standard | standard | standard | standard |
| Comparative product 1 | X | ◯ | ◯ | Δ |
| Comparative product 2 | X | ⊚ | ⊚ | Δ |
| Comparative product 3 | XX | ⊚ | ⊚ | Δ |
| Comparative product 4 | ⊚ | Δ | Δ | Δ |
| Product 1 of the | ⊚ | ◯ | ◯ | ◯ |
| present invention | | | | |
| Product 2 of the | ◯ | ⊚ | ⊚ | ⊚ |
| present invention | | | | |
| Product 3 of the | ◯ | ⊚ | ⊚ | ⊚ |
| present invention | | | | |

XX: apparently decreased,
X: slightly decreased,
Δ: equal,
◯: slightly increased,
⊚: apparently increased

According to the present invention, different protein-modifying effects from that of the case where TG or PG alone is used can be obtained and a food containing protein that has new characteristics can be produced. In addition, an optimum condition to treat substrate by TG and PG can be easily established. Therefore, the present invention is greatly useful for the food manufacturing field.

Numerous modifications and variations on the present invention are possible in light of the above teachings. It is, therefore, to be understood that within the scope of the accompanying claims, the invention may be practiced otherwise than as specifically described herein.

1. A method of modifying a protein comprising contacting said protein with a protein glutaminase and a transglutaminase for a time and under conditions where the protein glutaminase and transglutaminase act on said protein, wherein the protein glutaminase and the transglutaminase are added to said protein simultaneously or wherein said protein is contacted with the protein glutaminase before addition of the transglutaminse.

2. The method of claim 1, wherein a ratio by activity of the protein glutaminase to the transglutaminase ranges from 0.05:1 to 3:1.

3. The method of claim 1, wherein a ratio by activity of the protein glutaminase to the transglutaminase ranges from 0.05:1 to 2:1.

4. The method of claim 1, wherein a ratio by weight of the protein glutaminase to the transglutaminase ranges from 0.01:1 to 0.7:1.

5. The method of claim 1, wherein a ratio by weight of the protein glutaminase to the transglutaminase ranges from 0.01:1 to 0.4:1.

6. The method of claim 1, wherein a ratio by NMR signal intensity of the protein glutaminase to the transglutaminase ranges from 0.2:1 to 3.0:1.

7. The method of claim 1, wherein a ratio by NMR signal intensity of the protein glutaminase to the transglutaminase ranges from 0.2:1 to 2.3:1.

8. The method of claim 1, wherein the amount of the protein glutaminase ranges from 0.01 to 120 units per 1 gram-weight of protein.

9. The method of claim 1, wherein the amount of the transglutaminase ranges from 0.1 to 100 units per 1 gram-weight of protein.

10. The method of claim 1, wherein said protein is selected from the group consisting of a milk protein, whey protein, soybean protein, wheat gluten, muscle protein, plasma, collagen, and gelatin.

11. The method of claim 1, wherein said protein is in a food material.

12. The method of claim 11, wherein said food material is in the form of a raw material or a processed state.

13. The method of claim 12, wherein said food material is in a processed state and said processed state is selected from the group consisting of a sterilized state, a defatted state, a diluted state, a condensed state, and a dried state.

14. A food comprising a protein modified by the method of claim 1.

15. An enzyme preparation for modifying a protein comprising a protein glutaminase and a transglutaminase, wherein a ratio by activity of the protein glutaminase to the transglutaminase ranges from 0.05:1 to 3:1.

16. An enzyme preparation for modifying a protein comprising a protein glutaminase and a transglutaminase, wherein a ratio by weight of the protein glutaminase to the transglutaminase ranges from 0.01:1 to 0.7:1.

17. A enzyme preparation for modifying a protein comprising a protein glutaminase and a transglutaminase, wherein a ratio by NMR signal intensity of the protein glutaminase to the transglutaminase ranges from 0.2:1 to 3.0:1.

18. A method of establishing an optimum ratio of a protein glutaminase to a transglutaminase, both of which are to be added to a protein, comprising:
treating a protein with transglutaminase and protein glutaminase separately under presence of an isotope-labeled ammonium salt,
labeling a functional group of glutamine residue of the protein with the isotope, and
measuring an NMR signal intensity of the protein.

19. The method of claim 18, wherein said isotope is ¹⁵N.
